(19) United States
(12) Patent Application Publication
(10) Pub. No.: US 2009/0197830 A1
(43) Pub. Date: Aug. 6, 2009

(54) Combination of polychitosamine and hmg-coa reductase inhibitor for hyperlipidemia

(76) Inventor: Andre Aube, St-Sauveur (CA)

Correspondence Address: CHOATE, HALL & STEWART LLP, TWO INTERNATIONAL PLACE, BOSTON, MA, 02110, US

(21) Appl. No.: 11/662,891

(22) PCT Filed: Sep. 14, 2005
(86) PCT No.: PCT/CA2005/01406

§ 371 Date: Mar. 15, 2007

Related U.S. Application Data
(60) Provisional application No. 60/609,830, filed on Sep. 15, 2004.

(51) Int. Cl. A61K 31/722; A61P 3/06

(52) U.S. Cl. 514/55

(57) ABSTRACT

Combinations of therapeutic compounds for prophylaxis or treatment of hyperlipidemia and hyperlipidemia related disorders, such as hypercholesterolemia and the resultant atherosclerosis in a mammal. The combinations are useful for reducing serum cholesterol, and/or cholesteryl ester, triglycerides, phospholipids and fatty acids in a mammal. The methods of the preferred embodiments comprise administering to a mammal a first amount of polychitosamine and a second amount of an HMG-CoA reductase inhibitor (statin).

### CROSS-REFERENCE TO RELATED APPLICATIONS

This application claims the benefit under 35 U.S.C. § 119(e) of U.S. Provisional Application No. 60/609,830, filed Sep. 15, 2004.

### BACKGROUND OF THE INVENTION

1. Field of the Invention

The present invention relates to the field of therapeutic agents useful in lowering cholesterol or improving the ratio of HLD:LDL (particularly lowering low-density lipoproteins and/or increasing high density lipoproteins) and/or cholesteryl esters, triglycerides, phospholipids and fatty acids in a mammal, such as a human. More particularly, the invention relates to combination therapies, uses, and pharmaceutical compositions having greater therapeutic benefits than monotherapies using the same therapeutic substances.

2. Description of the Related Art

It is well known that hyperlipidemic conditions associated with elevated concentrations of total cholesterol and low-density lipoprotein (LDL) cholesterol are major risk factors for cardiovascular disease, such as atherosclerosis. Numerous studies have demonstrated that a low plasma concentration of high density lipoprotein (HDL) cholesterol (good cholesterol) is a powerful risk factor for the development of atherosclerosis (Barter and Rye, Atherosclerosis, 121, 1-12 (1996). HDL is one of the major classes of lipoproteins that function in the transport of lipids through the blood. The major lipids found associated with HDL include cholesterol, cholesteryl esters, triglycerides, phospholipids, and fatty acids. The other classes of lipoproteins found in the blood are low density lipoprotein (LDL), intermediate density lipoprotein (IDL), and very low density lipoprotein (VLDL). Since low levels of HDL cholesterol increase the risk of atherosclerosis, methods for elevating plasma HDL cholesterol would be therapeutically beneficial for the treatment of cardiovascular diseases, such as atherosclerosis. Cardiovascular diseases include, but are not limited to, coronary heart disease, peripheral vascular disease, and stroke.

One therapeutic approach to hyperlipidemic conditions has been the reduction of total cholesterol. Known use is made of the understanding that HMG-CoA reductase catalyzes the rate-limiting step in the biosynthesis of cholesterol (The Pharmacological Basis of Therapeutics, 9th ed., J. G. Hardman and L. E. Limberd, ed., McGraw-Hill, Inc., New York, pp. 884-888 (1996)). HMG-CoA reductase inhibitors (including the class of therapeutics commonly called “statins”) reduce blood serum levels of LDL cholesterol by competitive inhibition of this biosynthetic step (M. S. Brown, et al., J. Biol. Chem. 253, 1121-28 (1978)). Several statins have been developed or commercialized throughout the world. Atorvastatin calcium sold in North America under the brand Lipitor® is a potent reductase inhibitor. It is described in European Patent 409,281.

Warnings of side effects from use of HMG-CoA reductase inhibitors include liver dysfunction, skeletal muscle myopathy, rhabdomyolysis, and acute renal failure. Some of these effects are exacerbated when HMG-CoA reductase inhibitors are taken in greater doses. For example, a patient treated with 10 mg/day of Lipitor® may notice mild side effects. These side effects may greatly increase by simply raising the daily dose to 20 mg/day.

Furthermore, it has been shown that patients with well-controlled lipid profiles when treated at 10 mg/day may experience a return to elevated lipid profiles and require a dosage increase.

A number of combination therapies for the treatment of cardiovascular disease have been described in the literature. For example, a combination therapy of fluvastatin and niceritrol is described by J. Sasaki et al. (Int. J. Clin. Pharmacol. Ther., July; 33(7), 420-6 (1995)). These researchers conclude that the combination of fluvastatin with niceritrol “at a dose of 750 mg/day does not appear to augment or attenuate beneficial effects of fluvastatin.”

L. Cashin-Hemphill et al. (J. Am. Med. Assoc., 264 (23), 3013-17 (1990)) describe beneficial effects of a combination therapy of colestipol and niacin on coronary atherosclerosis.

A combination therapy of acipimox and simvastatin shows beneficial effects in patients having high triglyceride levels (N. Hoogerbrugge et al., J. Internal Med., 241, 151-55 (1997)).

The recently approved Vytorin® drug is commercialized by Schering Plough and Merck & Co. It combines simvastatin and ezetimibe in a single tablet and allows for a lower dose of simvastatin, without impacting the cholesterol lowering effect.

### SUMMARY OF THE INVENTION

However, none of the known combination therapies disclose the combination of a first amount of polychitosamine and a second amount of a HMG-CoA reductase inhibitor and concurrently achieve the benefits of the preferred embodiments.

The preferred embodiments improve efforts for preventing and/or treating hyperlipidemia, such as by reducing serum cholesterol, by providing a combination therapy approach and a novel pharmaceutical composition therefore.

An embodiment provides a pharmaceutical composition comprising: a) an HMG-CoA reductase inhibitor; and b) a polychitosamine.

An embodiment provides the use of the pharmaceutical composition of the present invention to increase the level of HDL in the blood of a mammal.

An embodiment provides a method for the prophylaxis or treatment of hyperlipidemia or hyperlipidemia-associated condition comprising administering to said patient: a) a first amount of a polychitosamine; and b) a second amount of an HMG-CoA reductase inhibitor; wherein the first and second amounts together comprise a therapeutically effective amount.

An embodiment provides a kit for the prophylaxis or treatment of hyperlipidemia or hyperlipidemia-associated condition in a mammal comprising a plurality of daily doses of dosage forms of an HMG-CoA reductase inhibitor, a plurality of daily doses of dosage forms of a polychitosamine together, and treatment regimen instructions.

### DETAILED DESCRIPTION OF THE PREFERRED EMBODIMENT

It has now been found that hyperlipidemic conditions in mammals may be effectively addressed by a combination of a first amount of polychitosamine and a second amount of a HMG-CoA reductase inhibitor. This combinatory approach has an important benefit of a milder side effect profile than HMG-CoA reductase inhibitor monotherapy at increased dosage levels. Effectiveness of a combination therapy is about equal to or better than increasing dosage levels of monotherapies of HMG-CoA reductase inhibitors.

As used herein the term “statin” or the term “HMG-CoA reductase inhibitor” or the term “HMG-CoA reductase inhibiting compound” refer to any entity derived from chemical or biological sources which may inhibit or decrease HMG-CoA reductase activity.

As used herein, “chitin” refers to a polymer formed primarily of repeating units of β (1-4) 2-acetamido-2-deoxy-D-glucose (or N-acetylglucosamine). Not every unit of naturally-occurring chitin is acetylated, with about 16% deacetylation.

As used herein, “chitosan” refers to chitin that has been partially or fully deacetylated. Chitosan is a polysaccharide formed primarily of repeating units of β (1-4) 2-amino-2-deoxy-D-glucose (or D-glucosamine). Further deacetylation of chitin can be achieved by processing of chitin. Deacetylation values can vary with chitin sources and with processing methods.

As used herein the term “polychitosamine” or the term “chitodextrine”, refers to a chitosan polymer having a molecular weight of less than about 650 kDa, preferably about 2-500 kDa, more preferably about 15-200 kDa, still more preferably about 20-100 kDa, yet more preferably about 25-60 kDa, and ideally about 30-50 kDa. In one embodiment, the molecular weight of the polychitosamine is about 30 kDa and in another embodiment, the molecular weight is about 30, 31, 32, 33, 34, 35, 36, 37, 38, 39, 40, 41, 42, 43, 44, 45, 46, 47, 48, 49, or 50 kDa. The polychitosamine can be obtained by cleaving a heavier molecular chain of chitosan. Preferably, polychitosamine is highly deacetylated, such as over about 80%, and more preferably over about 89%. Polychitosamine is understood herein to also encompass a chitosan salt formed from any chitosan molecule associated with a negatively charged anion. A series of anions has been used for that purpose. For example, anions derived from inorganic acids such as sulphuric acid (sulphate), phosphoric acid (phosphate), hydrochloric acid (chloride) and organic acids such as malic acid (malate), tartaric acid (tartrate), citric acid (citrate), succinic acid (succinate) and lactic acid (lactate), chitin, chitosan, and their derivatives.

As used herein, “nutraceuticals” is understood to encompass any ordinary food that has components or ingredients added to give a specific medical or physiological benefit other than a purely nutritional effect. It is also understood to include functional foods, dietary supplements and over the counter products sold without a prescription.

As used herein, “functional foods” is understood to encompass any food consumed as part of a usual diet that is similar in appearance to, or may be, a conventional food, and is demonstrated to have physiological benefits and/or reduce the risk of chronic disease beyond basic nutritional functions.

As used herein the term “combination therapy” refers to the administration of two or more therapeutic agents to treat a hyperlipidemic condition. Such administration encompasses co-administration of these therapeutic agents in a substantially simultaneous manner, such as in a single capsule having a fixed ratio of active ingredients or in multiple, separate dosage forms for each active agent. In addition, such administration also encompasses use of each type of therapeutic agent in a sequential or staggered manner. In either case, the treatment regimen will provide beneficial effects of the drug combination in treating the hyperlipidemic condition.

The phrase “therapeutically effective” is intended to qualify the combined amount of inhibitors in the combination therapy. This combined amount will achieve the goal of preventing, reducing or eliminating the hyperlipidemic condition.

The phrase “therapeutic compound” refers to a compound useful in the prophylaxis or treatment of a hyperlipidemic condition, such as, but not limited to, atherosclerosis, hypercholesterolemia, coronary heart disease, and cardiovascular disease including treatment of post heart attack patients in order to prolong survival 24 hours following myocardial infarction.

The combinations of the preferred embodiments will have a number of uses. For example, through dosage adjustment and medical monitoring, the individual dosages of the therapeutic compounds used in the combinations of the preferred embodiments will be lower than the typical dosages for the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Another use of the preferred embodiments will be in combinations having complementary effects or complementary modes of action. For example, HMG-CoA reductase inhibitors can control blood serum cholesterol levels by inhibiting an enzyme which is important in the biosynthesis of cholesterol. In contrast, polychitosamine can block the migration of cholesterol and/or other lipids such as cholesteryl esters and triglycerides from the intestinal tractus to the blood stream, and the reabsorption of bile acids from the intestinal tractus to the blood stream.

### Polychitosamine

Polychitosamine refers to a chitosan polymer having a molecular weight of less than about 650 kDa, preferably about 2-500 kDa, more preferably about 15-200 kDa, still more preferably about 20-100 kDa, yet more preferably about 25-60 kDa, and ideally about 30-50 kDa.

Chitosan is a naturally-occurring biopolymer that can also be obtained by partial or complete deacetylation of chitin that is the major component of the exoskeleton of shellfishes and insects. Chitosan is therefore a linear polymer composed of monomers of N-acetyl-2-amino-□-D-glucose and 2-amino-□-D-glucose. The presence of the primary amino groups of the 2-amino-□-D-glucose (D-glucosamine) units confers to chitosan its polycationic (positively charged) character that is neutralized by accompanying negatively charged anions. A series of anions has been used for that purpose. For example, anions derived from inorganic acids such as sulfuric acid (sulfate), phosphoric acid (phosphate), hydrochloric acid (chloride), and a mixture thereof and organic acids such as malic acid (malate), tartaric acid (tartrate), citric acid (citrate), lactic acid (lactate), acetic acid (acetate), formic acid (formate), glycolic acid (glycolate), oxalic acid, succinic acid, ascorbic acid, maleic acid, acrylic acid, gluconic acid, glutamic acid, propionic acid and a mixture thereof have been reported as salts of chitosan.

While there exists many extraction methods of the chitin from the crustacean shells, the principles of chitin extraction are relatively simple. In a certain treatment, the proteins are removed in a dilute solution of sodium hydroxide (such as about 1-10%) at high temperature (such as about 85-100° C.). Shells are then demineralized to remove calcium carbonate. This can be done by treating in a dilute solution of hydrochloric acid (1-10%) at room temperature. Depending on the severity of these treatments such as temperature, duration, concentration of the chemicals, concentration and size of the crushed shells, the physico-chemical characteristics of the extracted chitin can vary. For instance, three characteristics of the chitin, such as the degree of polymerization, acetylation, and purity, can be affected. Shell also contains lipids and pigments. Therefore, a decolorizing step is sometimes needed to obtain a white chitin. This can be done by soaking in organic solvents or in a very dilute solution of sodium hypochlorite. Again, these treatments can influence the characteristics of the chitin molecule.

Chitin can be deacetylated partially or totally. Such a deacetylated polymer is called chitosan. Chitosan compounds in a range of up to and exceeding 1×10⁶ molecular weight are derived commercially from chitin. In nature, chitosan is present in cell walls of Zygomycetes, a group of phytopathogenic fungi. Because of its significant content of free amino groups, chitosan has a markedly cationic character and has a positive charge at most pHs. Canadian Patent 2,085,292 discloses the hydrolysis of chitosan, the disclosure of which is incorporated herein by reference.

Polychitosamine can be produced by the process described in Canadian Patent 2,085,292, and recovered from solution using the process described in WO 2005/066213-A1 where the chitosan is salted out with a salting-out salt such as but not limited to sulfates, phosphates, citrates, nitrates, malates, tartrates, succinates, propionates, lactates and hydrogen phosphates. More preferably, these salting-out salts may be selected from the group consisting of: ammonium or sodium sulfate; sodium or potassium phosphates; sodium or potassium citrate; sodium tartrate; sodium malate; sodium nitrate; sodium lactate; sodium malonate; sodium succenate; sodium acetate; sodium propionate. Thus, the present invention includes any chitosan derivative obtained by any of the above-mentioned salts.

As an example, the citrate salt of chitosan can be illustrated as follows:

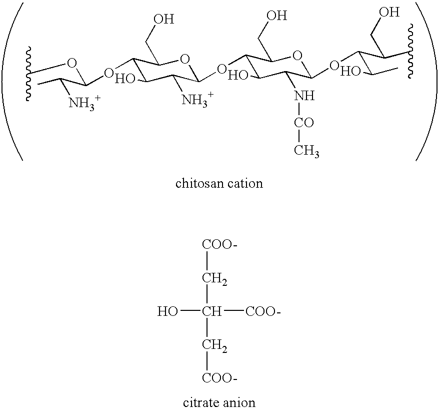

An approach for addressing hyperlipidemia is the use of polychitosamine.

In a mechanism of action, polychitosamine, in particular chitosan, can contain free amine groups which can attach themselves to lipids, such as cholesterol, via ionic bonds while in the intestinal tractus, forming an indissociable complex which is eventually excreted. Polychitosamine therefore can prevent lipids, such as cholesterol, from ever entering the bloodstream and adding to the total cholesterol content. Also, in reaction, the liver eliminates more cholesterol by using biliary acids. Therefore, there is elimination of both food cholesterol and that of biliary acids rich in cholesterol.

### Molecular Weight

Polychitosamine has many potential applications depending on its molecular weight. An average high molecular weight polychitosamine is about 650 kDa. Some applications are typical of medium or low molecular weight polychitosamine, ranging typically about 2-500 kDa. These applications include its use as an antifungal agent; a seed coating for improving crop yield; an elicitor of anti-pathogenic natural reactions in plants; a hypocholesterolemic agent in animals; an accelerator of lactic acid bacteria breeding; and a moisture-retaining agent for lotions, hair tonics and other cosmetics.

The molecular weight of polychitosamine is a feature that is particular to a certain application. The molecular weight of the native chitin has been reported to be as high as many million Daltons. However, chemical treatment tends to bring down the molecular weight of the polychitosamine, ranging from 100 KDa to 1500 KDa. Further treatment of the polychitosamine can lower the molecular weight even more. Low molecular weight could be produced by different ways including enzymatic or chemical methods. When the chain becomes shorter, the polychitosamine can be dissolved directly in water without the need of an acid. This is particularly useful for specific applications, such as in cosmetics or in medicine. Molecular weight of the polychitosamine can be measured by analytical methods, such as gel permeation chromatography, light scattering, or viscometry. Because of simplicity, viscometry is the most commonly used method.

In one embodiment, the molecular weight of the polychitosamine is about 30 kDa and in another embodiment, the molecular weight is about 30, 31, 32, 33, 34, 35, 36, 37, 38, 39, 40, 41, 42, 43, 44, 45, 46, 47, 48, 49, or 50 kDa. The polychitosamine can be obtained by cleaving a heavier molecular chain of chitosan.

### Deacetylation

Chitin can be deacetylated partially or totally. Naturally occurring chitin is acetylated, with about 16% deacetylation. Chitosan refers to chitin that has been partially or fully deacetylated. Chitosan is a polysaccharide formed primarily of repeating units of β (1-4) 2-amino-2-deoxy-D-glucose (or D-glucosamine). Further deacetylation of chitin can be achieved by processing of chitin. Deacetylation values can vary with chitin sources and with processing methods.

Since chitosan is made by deacetylation of chitin, the term degree of deacetylation (DAC) can be used to characterize chitosan. This value gives the proportion of monomeric units of which the acetylic groups that have been removed, indicating the proportion of free amino groups (reactive after dissolution in weak acid) on the polymer. DAC could vary from about 70 to about 100%, depending on the manufacturing method used. This parameter indicates the cationic charge of the molecule after dissolution in a weak acid. There are many methods of DAC measurements, such as UV and infrared spectroscopy, acid-base titration, nuclear magnetic resonance, dye absorption, and the like. Since there are no official standard methods, numbers tend to be different for different methods. In high value product, NMR can give a precise DAC number. However, titration or dye adsorption can serve as a quick and convenient method and yield similar results as NMR.

Chitin deacetylation towards chitosan can be obtained by various methods. The most used method is that of alkaline treatment (Horowitz, S. T. et al., 1957). With this method, around 80% of deacetylation can be achieved without significant decrease of molecular weight. A more intense deacetylation cannot be obtained by this method without a simultaneous uncontrolled decrease of the degree of polymerization. A more promising method is deacetylation by a thermo-mechano-chemical treatment (Pelletier et al., 1990). This method allows a more careful control of the various characteristics of the final product (average degree of polymerization and of deacetylation). Finally, a third method (Domard and Rinaudo, 1983) allows obtainment of a totally deacetylated product.

In a certain deacetylation protocol, when chitin is heated in a basic solution, such as a strong solution of sodium hydroxide (such as >about 40%) at high temperature (such as about 90-120° C.), chitosan is formed by deacetylation. This treatment can remove acetylic grouping on the amine radicals to a product (chitosan) that could be dissolved. It is said that at least 65% of the acetylic groups should be removed on each monomeric chitin to obtain the ability of being put in solution. The degree of deacetylation will vary according to the treatment conditions, such as duration, the temperature, and the concentration of the basic solution.

In the preferred embodiments, the polychitosamine has a deacetylation higher than about 80%. Preferably, the polychitosamine has a deacetylation higher than about 89%. More preferably, the polychitosamine has a deacetylation higher than about 89, 90, 91, 92, 93, 94, 95, 96, 97, 98, 99, or 100%. In a polychitosamine that has been deacetylated about 100%, the advantage being the polychitosamine forms a relatively homogeneous composition.

According to the present invention, the polychitosamine has a molecular weight of about 30 kDa and is deacetylated at least 80%. In a preferred embodiment, the polychitosamine has a molecular weight of about 30 kDa and is deacetylated at least 93% and is sold under the trademark Libracol® in Canada.

### HMG CoA Reductase Inhibitors

HMG-CoA reductase inhibitors encompassing a wide range of structures are useful in the combinations and methods of the preferred embodiments. Some HMG-CoA reductase inhibitors of particular interest in the preferred embodiments are Mevinolin, Mevastatin (U.S. Pat. No. 3,903,140), Nivastatin, Atorvastatin (EP 409281), Rosuvastatin, Lovastatin (U.S. Pat. No. 4,231,938), Simvastatin (U.S. Pat. No. 4,444,784), Pravastatin (U.S. Pat. No. 4,346,227), Fluvastatin (U.S. Pat. No. 4,739,073). Mevinolin is a naturally-occurring statin that is found in, for example oyster mushrooms and red yeast rice. The patents referenced are each herein incorporated by reference.

In a preferred embodiment, the statin is Atorvastatin. In a more preferred embodiment, the statin is Atorvastatin calcium, currently marketed under the name brand Lipitor®. In another preferred embodiment, the statin is Mevinolin. In yet another preferred embodiment, the statin is Rosuvastatin sold under the trademark Crestor®.

These therapeutic compounds can be used in the preferred embodiments in a variety of forms, including, but not limited to, acid form, salt form, racemates, enantiomers, zwitterions, and tautomers.

### Synergism

Synergy or synergism, most often refers to the phenomenon of two or more discrete influences or agents acting in common to create an effect which is greater than the sum of the effects each is able to create independently.

Warnings of side effects from use of HMG-CoA reductase inhibitors include liver dysfunction, skeletal muscle myopathy, rhabdomyolysis, and acute renal failure. Some of these effects are exacerbated when HMG-CoA reductase inhibitors are taken in greater doses. For example, a patient treated with 10 mg/day of Lipitor® may notice mild side effects. These side effects may greatly increase by simply raising the daily dose to 20 mg/day. Furthermore, it has been shown that patients with well-controlled lipid profiles when treated at 10 mg/day may experience a return to elevated lipid profiles and require a dosage increase.

Accordingly, an advantage of using drug synergism is a reduced amount of HMG-CoA reductase inhibitor administered to an individual, resulting in fewer side effects.

### Prevention and Treatment of Conditions

The preferred embodiments can be used to prevent, give relief from, or ameliorate a disease condition having hyperlipidemia as an element of a disease, such as atherosclerosis or coronary heart disease, or to protect against or treat further high cholesterol plasma or blood levels with the compounds and/or compositions of the preferred embodiments. The preferred embodiment may also be used to provide an aid to prolong survival to a patient, 24 hours following myocardial infraction. Hyperlipidemia is an elevation of lipids (fats) in the bloodstream. These lipids include cholesterol, cholesterol esters (compounds), phospholipids, triglycerides, and fatty acids. These lipids are transported in the blood as part of large molecules called lipoproteins.

Adverse effects of hyperlipidemia include atherosclerosis and coronary heart disease. Atherosclerosis is a disease characterized by the deposition of lipids, including cholesterol, in the arterial vessel wall, resulting in a narrowing of the vessel passages and ultimately hardening the vascular system. The primary cause of coronary heart disease (CHD) is atherosclerosis. CHD occurs when the arteries that supply blood to the heart muscle (coronary arteries) become hardened and narrowed. As a result of CHD, there could be angina or heart attack. Over time, CHD can weaken your heart muscle and contribute to heart failure or arrhythmias.

Hypercholesterolemia is also linked to cardiovascular disease. Cardiovascular disease refers to diseases of the heart and diseases of the blood vessel system (arteries, capillaries, veins) within a person's entire body, such as the brain, legs, and lungs. Cardiovascular diseases include, but are not limited to, coronary heart disease, peripheral vascular disease, and stroke.

Accordingly, the preferred embodiments may be used in preventing or treating hyperlipidemia and conditions associated with hyperlipidemia, such as hypercholesterolemia, atherosclerosis, coronary heart disease, and cardiovascular disease. The preferred embodiments also aid in prolonging survival to a post heart attack patient.

### Pharmaceutical Compositions

The compounds useful in the preferred embodiments can be presented with an acceptable carrier in the form of a pharmaceutical composition. The carrier is acceptable in the sense of being compatible with the other ingredients of the composition and is not deleterious to the recipient. The carrier can be a solid or a liquid, or both, and is preferably formulated with the compound as a unit-dose composition, for example, a capsule or tablet, which can contain from about 0.05% to about 95% by weight of the active compound. Examples of suitable carriers, diluents, and excipients include, but are not limited to, lactose, dextrose, sucrose, sorbitol, mannitol, starches, gum acacia, alginates, tragacanth, gelatin, calcium silicate, cellulose, magnesium carbonate, or a phospholipid with which the polymer can form a micelle. Other pharmacologically active substances can also be present, including other compounds of the preferred embodiments. For example, more than one statin may be used together. The pharmaceutical compositions of the preferred embodiments can be prepared by any of the well-known techniques of pharmacy, comprising admixing the components.

In practicing the methods of the preferred embodiments, administration of the HMG-CoA reductase inhibitor, in combination therapy, may be accomplished by oral route.

For oral administration, compounds used in the combination therapy can be in the form of, for example, but not limited to, a tablet, capsule, suspension, powders (e.g., for sprinkling or food), or liquid. Other embodiments include sustained-release capsules, enteric coated tablets, soft gel capsules, and other sustained-release technologies. Capsules, tablets, liquid, or powders, and the like can be prepared by conventional methods well-known in the art. The compounds are preferably made in the form of a dosage unit containing a specified amount of the compound. Examples of dosage units are tablets or capsules.

Pharmaceutical compositions for use in the treatment methods of the preferred embodiments can be administered in oral form for compounds of the composition, or by parenteral, such as intravenous administration for the HMG-CoA reductase inhibitor and oral administration of the polychitosamine. For convenience, oral administration of both therapeutic substances is preferred. Dosing for oral administration can be with a regimen calling for single daily dose, or for a single dose every other day, or for multiple, spaced doses throughout the day.

### Nutraceuticals

The compounds useful in the preferred embodiment can be incorporated in a functional food or nutraceutical. These compounds may be presented in the form of active agents such as cholesterol lowering agents. As such, these compounds may be useful in the manufacture of nutraceuticals and/or functional foods useful for preventing hyperlipidemia associated conditions.

In a preferred embodiment, the polychitosamine and statin compounds are incorporated in functional foods including but not limited to: beverages, including but not limited to sodas, water, sports/energy drinks, canned and bottled juices, fresh and refrigerated juices, frozen juices, yoghurt drinks, smoothies, teas and coffees; breads and grains, including but not limited to breakfast cereals, breads, baked goods, baking ingredients such as flour, frozen breads, dried breads and crackers, pastas; snack foods, including but not limited to nutrition bars, weight loss bars, energy/sports bars, candy bars, chips, gum; packaged and prepared foods, including but not limited to frozen foods such as pizzas and dinners, canned and dried soups, desserts including cookies; condiments, including but not limited to dressings, spreads, sauces; dairy and dairy alternatives, including but not limited to milk, cheese, butter, ice cream, yoghurt, margarine and soymilk.

### Dosages

A total daily dose of an HMG-CoA reductase inhibitor can generally be in the range of from about 0.1 to about 100 mg/day in single or divided doses. Lovastatin, Atorvastatin, or Mevastatin, for example are generally each administered separately in a daily dose of about 10 to about 60 mg/day. Fluvastatin is generally administered in a daily dose of about 20 to about 40 mg/day. In an embodiment, HMG-CoA reductase inhibitor is administered at about 10 mg per day.

In the case of pharmaceutically acceptable statin salts, the weights indicated above refer to the weight of the acid equivalent or the base equivalent of the therapeutic compound derived from the salt.

For a polychitosamine, a total daily dose of about 400 mg to about 4.8 grams per day and preferably between about 800 mg and about 2.4 grams per day may generally be appropriate. In an embodiment, polychitosamine is administered at about 400 mg per day. The polychitosamine is preferably taken three times a day. The polychitosamine is preferably taken with meals.

### Certain Dosages

As used herein, the term “total daily dose” refers to the amount of composition administered to an individual in one day. The term “dose” refers to the amount of composition administered to an individual at one time. The term “unit dose” refers to the amount of a composition pre-packaged by the manufacturer or pharmacist in standardized amounts. Thus, for example, the dose of ingredients in a single tablet or capsule would be considered a single “unit dose” whether one or more tablets or capsules are taken simultaneously.

In another embodiment, a polychitosamine is administered with a total daily dose of about 600 mg to about 2400 mg and a statin is administered at a total daily dose of about 6 mg to about 80 mg. Preferably, the total daily dose is administered once per day in about 1 to about 3 unit doses, preferably in capsule form. Accordingly, each unit dose may contain about 200 mg to about 1200 mg of polychitosamine. Also, each unit dose may contain about 2 mg to about 80 mg of statin. In an embodiment, the total daily dose is administered in two unit doses administered once a day; the unit dose containing about 600 mg of polychitosamine and about 5 mg of statin. The dose is preferably administered with meals.

In another embodiment, the total daily dose is administered in two doses per day. In each dose, there is a polychitosamine in an amount of about 200 mg to about 1200 mg and a statin in an amount of about 3 mg to about 40 mg. Preferably, the total daily dose is administered in two doses per day with about 1 to about 2 unit doses per dose. Accordingly, each unit dose may contain about 200 mg to about 600 mg of polychitosamine. Also, each unit dose may contain about 2 mg to about 40 mg of statin. In an embodiment, the unit dose is administered in one capsule administered twice a day; the unit dose containing about 600 mg of polychitosamine and about 5 mg of statin. The dose is preferably administered with meals.

Certain embodiments with regard to approximate dosage are shown in Table 1.

TABLE 1

| | |
|---|---|
| Frequency of | Approximate Dosage per administration |

| administration | Polychitosamine | Lovastatin | Simvastatin | Pravastatin |
|---|---|---|---|---|
| Once per day | 600-2400 mg | 12-80 mg | 6-80 mg | 6-40 mg |
| (1-3 unit doses) | | | | |
| Per unit dose | 200-800 mg | 4-80 mg | 2-80 mg | 2-40 mg |
| Twice per day | 400-1200 mg | 6-40 mg | 3-40 mg | 3-20 mg |
| (1-2 unit doses) | | | | |
| Per unit dose | 200-800 mg | 3-40 mg | 1.5-40 mg | 1.5-20 mg |

| Frequency of | Dosage per administration | | | | |
|---|---|---|---|---|---|
| administration | Polychitosamine | Fluvastatin | Atorvastatin | Rosuvastatin | Mevinolin |
| Once per day | 600-2400 mg | 12-80 mg | 6-80 mg | 6-40 mg | 3-80 mg |
| (1-3 capsules) | | | | | |
| Per capsule | 200-800 mg | 4-80 mg | 2-80 mg | 2-40 mg | 1-80 mg |
| Twice per day | 400-1200 mg | 6-40 mg | 3-40 mg | 3-20 mg | 2-40 mg |
| (1-2 capsules) | | | | | |
| Per capsule | 200-800 mg | 3-40 mg | 1.5-40 mg | 1.5-20 mg | 1-40 mg |

The daily doses described in the preceding paragraphs, for the various therapeutic compounds can be administered to the patient in a single dose, or in proportionate multiple subdoses. Subdoses can be administered about 2 to about 3 times per day. Doses can be in sustained-release form effective to obtain desired results.

The dosage regimen to treat hyperlipidemia and hyperlipidemia-associated conditions, and reduce plasma cholesterol with the combination therapy and pharmaceutical compositions of the preferred embodiments is selected in accordance with a variety of factors. These factors include, but are not limited to, the type, age, weight, sex, diet, and medical condition of the patient, the severity of the disease, the route of administration, pharmacological considerations, such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Initial treatment of a patient suffering from a hyperlipidemic condition, such as, but not limited to, hypercholesterolemia, atherosclerosis, coronary heart disease, and cardiovascular disease, can begin with the dosages indicated above. Treatment should generally be continued as necessary over a period of several weeks to several months or years until the condition has been controlled or eliminated. Patients undergoing treatment with the compounds or compositions disclosed herein can be routinely monitored by, for example, measuring serum LDL and total cholesterol levels by any of the methods well known in the art, to determine the effectiveness of the combination therapy.

### Kits

The preferred embodiments also relate to kits for conveniently dispensing the combination therapy. These kits will preferably contain a plurality of daily doses of the HMG-CoA reductase inhibitor and of the polychitosamine. The daily doses are preferably separate dosage forms of each medicament. Instructions are also provided for patient and/or dispensing physician or pharmacist. The kits may contain supplies for a given time duration of treatment such as one month.

Blister package with 7 days worth of treatment, each day indicated (Monday, Tuesday, Wednesday, Thursday, Friday, Saturday, Sunday). Each day has two capsules or tablets, one indicated as “breakfast”, the other indicated as “supper”. There is a total of 14 capsules per blister pack (2 rows and 7 columns) and four blisters in a box—enough for a four week (28 day) supply. Alternatively, you could have one single row for once daily dosing (dinner only), or three rows for three times per day dosing (breakfast, lunch, dinner). According to this embodiment, the statin and the polychitosamine are contained in the same capsule or tablet.

According to another embodiment, the statin and the polychitosamine are in separate tablets or capsules, one dose per day of each, for each of seven days. This would represent 2 rows and 7 columns per blister, with 4 blisters per box.

Those skilled in the art will know, or be able to ascertain, using no more than routine experimentation, many equivalents to the specific embodiments of the invention described herein. These and all other equivalents are intended to be encompassed by the following claims.

What is claimed is:

1. A pharmaceutical composition comprising:
a) an HMG-CoA reductase inhibitor; and
b) a polychitosamine.

2. The pharmaceutical composition of claim 1, further comprising a pharmaceutically acceptable carrier.

3. The pharmaceutical composition of claim 1, characterized in that the HMG-CoA reductase inhibitor is selected from the group consisting of Lovastatin, Simvastatin, Pravastatin, Fluvastatin, Atorvastatin, Mevinolin, Rosuvastatin, Nivastatin, calcium Atorvastatin and Mevastatin.

4. The pharmaceutical composition of claim 3, characterized in that the HMG-CoA reductase inhibitor is Atorvastatin.

5. The pharmaceutical composition of claim 3, characterized in that the HMG-CoA reductase inhibitor is calcium Atorvastatin.

6. The pharmaceutical composition of claim 5, characterized in that the HMG-CoA reductase inhibitor is Lipitor®.

7. The pharmaceutical composition of claim 3, characterized in that the HMG-CoA reductase inhibitor is Rosuvastatin.

8. The pharmaceutical composition of claim 7, characterized in that the HMG-CoA reductase inhibitor is Crestor®.

9. The pharmaceutical composition of claim 3, characterized in that the HMG-CoA reductase inhibitor is Mevinolin.

10. The pharmaceutical composition of claim 3, characterized in that the HMG-CoA reductase inhibitor is Nivastatin.

11. The pharmaceutical composition of claim 3, characterized in that the HMG-CoA reductase inhibitor is Simvastatin.

12. The pharmaceutical composition of claim 11, characterized in that the HMG-CoA reductase inhibitor is Zocor®.

13. The pharmaceutical composition of claim 1, characterized in that the polychitosamine has a molecular weight of about 30 kDa and is deacetylated at least about 93%.

14. The pharmaceutical composition of claim 1, characterized in that the polychitosamine is Libracol®.

15. The pharmaceutical composition of claim 1, characterized in that the polychitosamine has a molecular weight ranging between 35 and 50 kDa.

16. The pharmaceutical composition of claim 15, characterized in that the polychitosamine has a molecular weight of about 40 kDa.

17. The pharmaceutical composition of claim 16, characterized in that the polychitosamine is HEP40®.

18. The pharmaceutical composition of claim 1, characterized in that the therapeutically effective amount of the HMG-CoA reductase inhibitor is about 6 mg per day.

19. The pharmaceutical composition of claim 1, characterized in that the therapeutically effective amount of the polychitosamine is at least about 400 mg per day.

20. The pharmaceutical composition of claim 1, characterized in that the therapeutically effective amount of the HMG-CoA reductase inhibitor is about 6 mg to about 80 per day and wherein the therapeutically effective amount of the polychitosamine is about 600 mg to about 2400 mg per day.

21. A method for the prophylaxis or treatment of hyperlipidemia or hyperlipidemia-associated condition comprising administering to said patient:
a) a first amount of a polychitosamine; and
b) a second amount of an HMG-CoA reductase inhibitor;
wherein the first and second amounts together comprise a therapeutically effective amount.

22. The method of claim 18, characterized in that the hyperlipidemia-associated condition is selected from the group consisting of hypercholesterolemia, atherosclerosis, coronary heart disease, cardiovascular disease and post heart attack recovery.

23. The method of claim 18, characterized in that the HMG-CoA reductase inhibitor is selected from the group consisting of Mevinolin, Lovastatin, Simvastatin, Pravastatin, Fluvastatin, Atorvastatin, Rosuvastatin, Nivastatin, calcium Atorvastatin and Mevastatin.

24. The method of claim 18, characterized in that the HMG-CoA reductase inhibitor is Atorvastatin.

25. The method of claim 18, characterized in that the HMG-CoA reductase inhibitor is calcium Atorvastatin.

26. The method of claim 20, characterized in that the HMG-CoA reductase inhibitor is Lipitor®.

27. The method of claim 18, characterized in that the HMG-CoA reductase inhibitor is Rosuvastatin.

28. The method of claim 27, characterized in that the HMG-CoA reductase inhibitor is Crestor®.

29. The method of claim 23, characterized in that the HMG-CoA reductase inhibitor is Mevinolin.

30. The method of claim 23, characterized in that the HMG-CoA reductase inhibitor is Nivastatin.

31. The method of claim 23, characterized in that the HMG-CoA reductase inhibitor is Simvastatin.

32. The method of claim 31, characterized in that the HMG-CoA reductase inhibitor is Zocor®.

33. The method of claim 23, characterized in that the polychitosamine has a molecular weight of about 30 kDa and is deacetylated at least about 93%.

34. The method of claim 21, characterized in that the polychitosamine is Libracol®.

35. The method of claim 21, characterized in that the polychitosamine has a molecular weight ranging between 35 and 50 kDa.

36. The method of claim 21, characterized in that the polychitosamine has a molecular weight of about 40 kDa.

37. The method of claim 36, characterized in that the polychitosamine is HEP40®.

38. The method of claim 21, characterized in that the therapeutically effective amount of the HMG-CoA reductase inhibitor is at least about 6 mg per day.

39. The method of claim 21, characterized in that the therapeutically effective amount of the polychitosamine is at least about 400 mg per day.

40. The method of claim 21, characterized in that the therapeutically effective amount of the HMG-CoA reductase inhibitor is about 6 mg to about 80 per day and wherein the therapeutically effective amount of the polychitosamine is about 600 mg to about 2400 mg per day.

41. The method of claim 40, characterized in that the therapeutically effective amounts of the HMG-CoA reductase inhibitor and the therapeutically effective amount of the polychitosamine are administered once a day.

42. The method of claim 40, characterized in that the therapeutically effective amounts of the HMG-CoA reductase inhibitor and the therapeutically effective amount of the polychitosamine are administered twice a day.

43. A kit for the prophylaxis or treatment of hyperlipidemia or hyperlipidemia-associated condition in a mammal comprising a plurality of daily doses of dosage forms of an HMG-CoA reductase inhibitor, a plurality of daily doses of dosage forms of a polychitosamine together, and treatment regimen instructions.

44. The kit of claim 43, characterized in that the plurality of daily doses comprises separate daily doses of the HMG-CoA reductase inhibitor and separate daily doses of the polychitosamine.

45. The kit of claim 43, characterized in that the HMG-CoA reductase inhibitor is provided in dose units ranging from 2 mg to 80 mg.

46. The kit of claim 43, characterized in that the polychitosamine is provided in dose units ranging from 200 mg to 1200 mg.
